(19) United States
(12) Patent Application Publication
(10) Pub. No.: US 2013/0041451 A1
(43) Pub. Date: Feb. 14, 2013

(54) PROSTHESIS DEPLOYMENT SYSTEM FOR OPEN SURGICAL REPAIR

(71) Applicants: Donald F. Patterson, Bloomington, IN (US); Laura A. Boehm, Hamilton, OH (US); Morgan K. T. Humphrey, Owensboro, KY (US); Jeremy E. Phillips, Glasgow, KY (US); Sally A. Zimmerman, Ft. Thomas, KY (US)

(72) Inventors: Donald F. Patterson, Bloomington, IN (US); Laura A. Boehm, Hamilton, OH (US); Morgan K. T. Humphrey, Owensboro, KY (US); Jeremy E. Phillips, Glasgow, KY (US); Sally A. Zimmerman, Ft. Thomas, KY (US)

(73) Assignee: Cook Medical Technologies LLC, Bloomington, IN (US)

(21) Appl. No.: 13/206,078

(22) Filed: Aug. 9, 2011

(51) Int. Cl. A61F 2/84

(52) U.S. Cl. 623/1.12

(57) ABSTRACT

A deployment system for open surgical repair of a body vessel is provided. The system includes a prosthesis retained in a compressed configuration by a retainer sheath. A splitting member can include a portion disposed internally within the retainer sheath and a portion accessible from at least one of the outer ends of the retainer sheath. Retraction of the accessible portion toward a middle of the prosthesis can split the wall of the retainer sheath to allow for expansion of a segment of the prosthesis for engagement with a first portion of the body vessel. Another segment of the prosthesis is expanded for engagement with a second portion in order for the prosthesis to interconnect the first and second portions of the body vessel. A barrier segment may be disposed within the retainer sheath between the splitting member and the prosthesis.

### BACKGROUND

The present disclosure relates generally to medical prosthesis deployment systems for open surgical repair. More particularly, the present disclosure relates to a deployment system for a prosthesis to open surgical repair a transected body vessel for gaining hemostasis during emergency medical procedures.

Trauma physicians frequently encounter patients having traumatic injury to a body vessel, such as lacerated vessels or even transected vessels, resulting from gunshots, knife wounds, motor vehicle accidents, explosions, etc. Significant damage to a body vessel may expose a patient to deleterious conditions such as the loss of a limb, loss of function of a limb, increased risk of stroke, impairment of neurological functions, and compartment syndrome, among others. Particularly severe cases of vascular injury and blood loss may even result in death. In such severe situations, the immediate goal is to obtain hemostasis while maintaining perfusion of adequate blood flow to critical organs, such as the brain, liver, kidneys, and heart.

Examples of treatment that are commonly performed by trauma physicians to treat body vessel injuries include clamping the vessel with a hemostat, use of a balloon tamponade, ligation of the damaged vessel at or near the site of injury, or the insertion of one or more temporary shunts. However, conventional surgical repair is generally difficult with such actively bleeding, moribund patients. In many instances, there is simply not enough time to repair the body vessel adequately by re-approximating and suturing the body vessel. In many situations, the trauma physician will simply insert a temporary shunt (such as a Pruitt-Inahara Shunt) into the vessel. However, use of temporary shunts has been linked to the formation of clots. This may require returning the patient to the operating room for treatment and removal of the clots, often within about 36 to 48 hours of the original repair. Since shunts are generally placed as a temporary measure to restore blood flow and stop excessive blood loss, the shunt is typically removed when the patient has stabilized (generally a few days later) by a specialized vascular surgeon. After removal, the vascular surgeon will replace the shunt with a vascular graft, such as a fabric graft that is sewn into place. Ligation of the damaged blood vessel may result in muscle necrosis, loss of muscle function, or a potential limb loss or death.

Due to the nature of the body vessel injury that may be encountered, the use of shunts, repairing and/or ligating of a blood vessel often requires that such treatments be rapidly performed at great speed, and with a high degree of physician skill. Such treatments may occupy an undue amount of time and attention of the trauma physician at a time when other pressing issues regarding the patient's treatment require immediate attention. In addition, since the level of particularized skill required may exceed that possessed by the typical trauma physician, particularly traumatic episodes may require the skills of a physician specially trained to address the particular trauma, such as a vascular trauma, and to stabilize the patient in the best manner possible under the circumstances of the case.

Some open surgical techniques utilize sutures to affix damaged tissue portions surrounding fittings that have been deployed with the vessel, which requires the trauma physician to take time to tie the sutures properly. Although in modern medicine sutures can be tied in relatively rapid fashion, any step in a repair process that occupies physician time in an emergency situation is potentially problematic. In addition, the use of sutures to affix the vessel to the fitting compresses the tissue of the vessel against the fitting. Compression of tissue may increase the risk of necrosis of the portion of the vessel tissue on the side of the suture remote from the blood supply. When present, necrosis of this portion of the vessel tissue may result in the tissue separating at the point of the sutures. In this event, the connection between the vessel and the fitting may eventually become weakened and subject to failure. If the connection fails, the device may disengage from the vessel. Therefore, efforts continue to develop techniques that reduce the physician time required for such techniques, so that this time can be spent on other potentially life-saving measures.

It would be desirable to provide a prosthesis deployment system for use in open surgical repair of an injured body vessel, such as an artery or a vein, (and in particular a transected vessel) during emergency surgery in a manner that is time effective, that addresses the trauma at hand to the extent possible, and that utilizes techniques that may be readily practiced by an trauma physician.

### SUMMARY

In one embodiment, a deployment system for repair of a body vessel is provided. The system can include at least one retainer sheath fitted at least partially over a segment of a prosthesis to retain the segment in a compressed configuration. The prosthesis has a first prosthesis end and a second prosthesis end, and is radially movable between a compressed configuration and an expanded configuration. The retainer sheath is a tubular body having a first sheath end, a second sheath end, and a lumen extending therethrough to receive the prosthesis. At least one splitting member can have an internal portion disposed between a luminal wall of the retainer sheath and the prosthesis, and an external portion disposed external to the retainer sheath. The splitting member is operable to split a wall of the retainer sheath along a direction toward a middle of the prosthesis away from at least one of the first and second sheath ends upon retraction of the external portion of the splitting member. In response to being split by the splitting member, the retainer sheath has a split configuration and the corresponding prosthesis end is allowed to move to the expanded configuration for engagement with a body vessel wall.

In one aspect, at least one inner barrier segment is disposed between the internal portion of the splitting member and an outside wall of the prosthesis. The barrier segment can extend axially at least partially between the first and second sheath ends. The barrier segment may have a segment width along the circumference of the prosthesis that is in the expanded configuration, and the retainer sheath in the split configuration may have a sheath width along the circumference of the prosthesis that is in the expanded configuration. The segment width and the sheath width can be dimensioned and arranged to permit at least one open circumferential area between the barrier segment and the retainer sheath and allow direct contact between the prosthesis in the expanded configuration and the body vessel wall.

In another embodiment, a method of interconnecting a first vessel portion and a second vessel portion of a transected body vessel is provided. The method can include one or more of the following steps, such as introducing a first end of a prosthesis retained in a compressed configuration by a retainer sheath in a first vessel portion. A splitting member is associated with the retainer sheath and is operable to split a wall of the retainer sheath. A portion of the splitting member can be retracted in a direction away from a middle of the prosthesis to split the retainer sheath from the outer end and toward the middle such that the first end of the prosthesis is permitted to expand to an expanded configuration for engagement with a vessel wall of the first vessel portion. A second end of the prosthesis retained in a compressed configuration by the same retainer sheath or a second retainer sheath can be introduced in a second vessel portion. A second splitting member is associated with the second retainer sheath and is operable to split a wall of the second retainer sheath. A portion of the second splitting member can be retracted in a direction away from a middle of the prosthesis to split the retainer sheath from the outer end and toward the middle such that the second end of the prosthesis is permitted to expand to an expanded configuration for engagement with a vessel wall of the second vessel portion.

### BRIEF DESCRIPTIONS OF THE DRAWINGS

FIG. 1 is a perspective view of one example of a deployment system for vascular repair of a body vessel.

FIG. 2 is a side cross-sectional view of an end of a deployment system for vascular repair of a body vessel.

FIG. 3 is a transverse sectional view a deployment system for vascular repair of a body vessel.

FIG. 4A is a perspective view of another example of a deployment system for vascular repair of a body vessel.

FIG. 4B is a perspective view of the deployment system of FIG. 4A, depicting a splitting member performing a cutting action.

FIG. 5 is a longitudinal transverse sectional view of the deployment system of FIG. 4.

FIG. 6 is cross-sectional view of the deployment system, taken along lines 6-6 in FIG. 4.

FIG. 7A is cross-sectional view of the deployment system of FIG. 4.

FIG. 7B is cross-sectional view of the deployment system of FIG. 4, after expansion of the prosthesis within a body vessel.

FIG. 8 is a perspective view of another example of a deployment system for vascular repair of a body vessel.

FIGS. 9-10 are perspective views of one example of a retainer sheath used in a deployment system.

FIGS. 11A-11F are partial side views depicting a method of using a deployment system.

FIG. 12A is cross-sectional view of a deployment system having a plurality of splitting members.

FIG. 12B is cross-sectional view of the deployment system of FIG. 12A, after expansion of the prosthesis within a body vessel.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. It should also be noted that in the Figures like-referenced numerals designate corresponding components throughout the different views.

The prosthesis delivery systems described herein can be useful for open surgical repair of a body vessel, such as a blood vessel, during a medical procedure such as an emergency open surgical procedure. The prosthesis deployment systems can be particularly useful to deliver a prosthesis for repair of a lacerated artery or vein during emergency surgery, and particularly, to obtain hemostasis while maintaining blood perfusion, especially after transection of the body vessel.

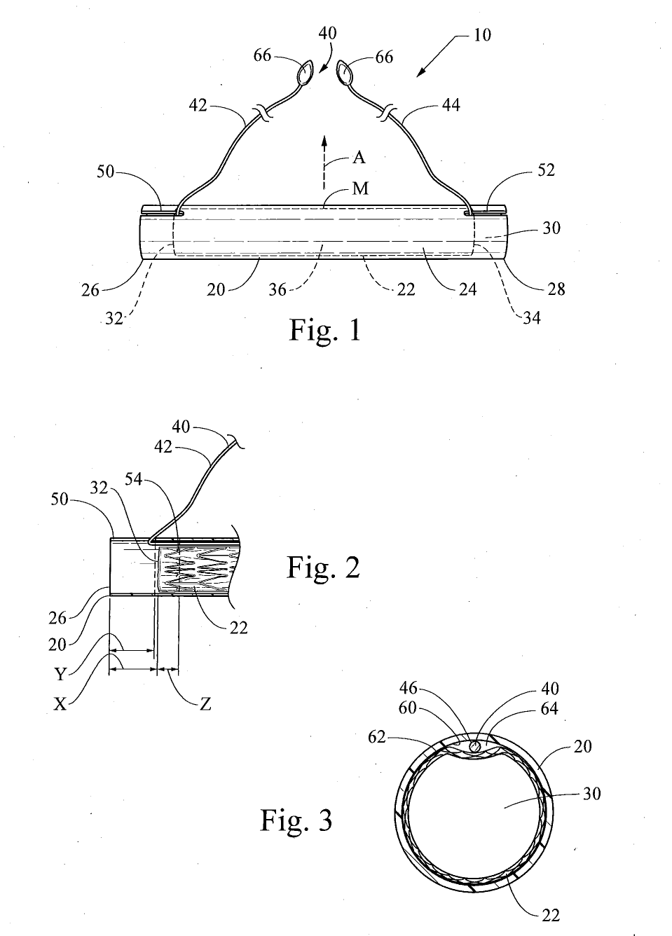

FIG. 1 depicts one example of a deployment system 10 for vascular repair of a body vessel. Deployment system 10 can include one or more outer retainer sheaths, such as an outer retainer sheath 20, and one or more splitting members 40. The retainer sheath 20 can be fitted over a prosthesis 22 (shown in dashed lines). The retainer sheath can be in a non-split configuration to retain portions of the prosthesis in a radially compressed configuration for delivery into the body vessel. The retainer sheath 20 can include a tubular body 24 extending between a first end 26 and a second end 28. A lumen 30 extends through the retainer sheath 20 and is sized to receive the prosthesis 22 in the compressed configuration. The prosthesis 22 has a first outer end 32 and a second outer end 34 each configured to engage the wall of a body vessel portion, and an intermediate segment 36 between the first and second outer ends 32, 34. The intermediate segment 36 may be positioned within the body vessel to remain at least partially outside the vessels portions.

The splitting member 40 can have a first end portion 42 and a second end portion 44 extending outwardly from the first and second ends 26, 28 of the retainer sheath 20, respectively. The first and/or second end portions 42, 44 of the splitting member 40 can be retracted toward a middle M of the prosthesis 22 from the first and second ends 26, 28 and may be further retracted outwardly away from the middle M in a radial direction of arrow A. When retracted, the splitting member 40 can split or cut through the wall of the retainer sheath 20 in an outside-in direction. The retainer sheath can be in a split configuration to allow for expansion of the outer ends 32, 34 of the prosthesis 22 to a radially expanded configuration for engagement with a body vessel wall before the expansion of the intermediate segment 36 of the prosthesis 22. In one example, the first and second end portions 42, 44 of the splitting member can be retracted together for simultaneous expansion of the outer ends 32, 34 of prosthesis 22, or alternatively, can be retracted separately for sequential expansion of the outer ends of the prosthesis.

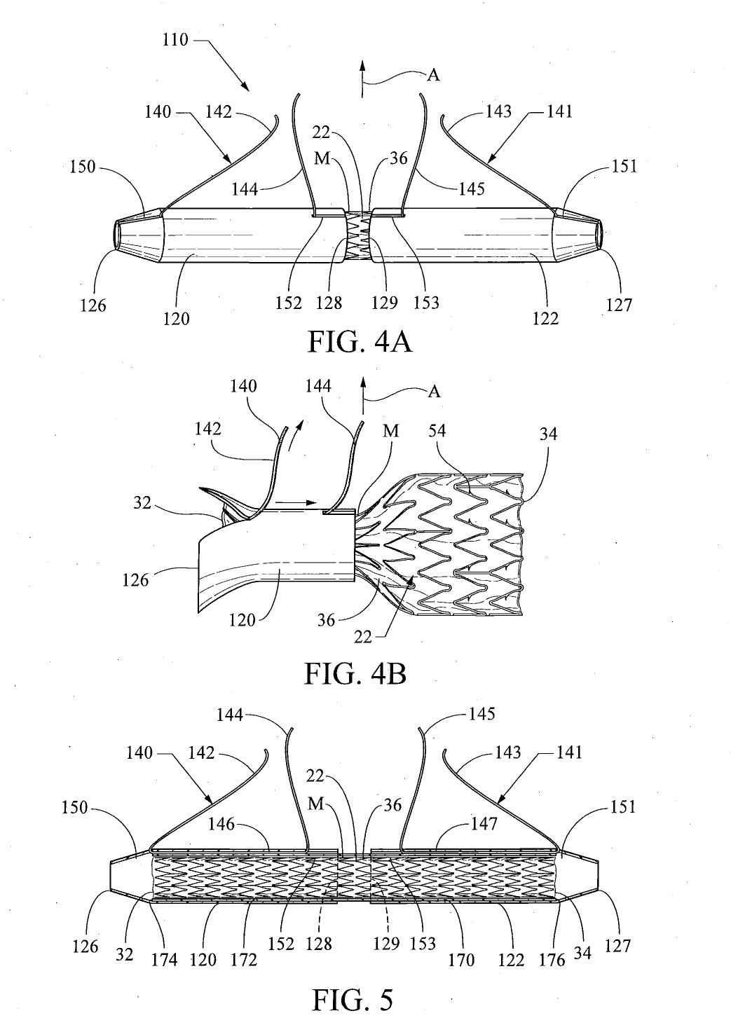

The axial length of the retainer sheath 20 and the prosthesis 22 can be coextensive or different from each other. For example, FIGS. 1-2 depict the retainer sheath 20 having an axial length so that its ends 26, 28 extend outwardly beyond the outer ends 32, 34 of the prosthesis 22 by a distance X, such as, e.g., about 1 cm. This arrangement can allow the outer end of the retainer sheath to be conformable into a smaller profile for insertion into the vessel portion. The first and second ends 26, 28 of the retainer sheath 20 that are extended beyond the outer ends 32, 34 of the prosthesis 22 can be tapered ends, as shown in FIG. 4A, to facilitate introduction into an end opening of the body vessel portion.

In one example, the retainer sheath 20 can have at least one slit formed therein to allow the passage of the splitting member 40 and guide the splitting action with the splitting member. In FIGS. 1-2, a first slit 50 and a second slit 52, e.g., shaped as an axial slit, can be formed in the wall of the retainer sheath 20 to extend from the ends 26, 28 of the retainer sheath inward by a length Y. The length Y may terminate at a position to correspond at least to the outer end of the prosthesis, although can terminate short of the outer end of the prosthesis to ensure that a ring of sheath material surrounds the prosthesis for facilitating its compressed configuration. Alternatively, the length of the slit may terminate beyond the outer end of the prosthesis to a position farther inward with respect to an anchoring member 54 of the prosthesis 22, which is disposed a length Z inward from the outer ends of the prosthesis, as shown in FIG. 2. Such inward arrangement can permit at least a portion of the anchoring member to have direct access to engage the vessel wall when the retainer sheath is split. The slits may provide a guiding edge for the splitting member 40 when being retracted. The slit may also permit the retainer sheath ends to be conformable into a smaller profile for insertion into the vessel portion.

In FIG. 3, a segment, such as an intermediate portion 46 of the splitting member 40, can be disposed within the lumen 30 of the retainer sheath 20, sandwiched between the luminal wall 60 of the retainer sheath and the outer surface 62 of the prosthesis 22. The intermediate portion 46 of the splitting member is disposed between the first and second end portions 42, 44 and can extend axially within an annular space 64 between the retainer sheath and the prosthesis. The splitting member can have a tensile strength sufficient to be pulled during the splitting action without breaking. The first and second end portions and the intermediate portion of the splitting member can be formed from different materials that are fixedly attached to one another by an attachment mechanism such as welding, soldering, bonding, or other known attachment mechanisms. Optionally, the first and second end portions and the intermediate portion of the splitting member can be formed from the same material, and preferably integrally as a single unit. The splitting member 40 may be formed generally as a flexible elongated member such as, e.g., a metal wire, plastic strip, a suture, or the like. In one example, the splitting member is a stainless steel, copper, or nitinol wire having a diameter of about 0.25 mm (0.01 inches). According to FIG. 1, the ends of the splitting member 40 can be an enlarged end 66 for improved grippability by the end user during retraction of the splitting member. The first end portion, the second end portion, or both, may be retracted to split the retainer sheath.

FIG. 4A depicts another example of a deployment system 110 for vascular repair of a body vessel, which includes a pair of the systems 10 on each outer end of the prosthesis for independently controlling the expansion at each end. It is contemplated that more than two systems 10 can be provided for controlling the expansion along different segments of the prosthesis. Deployment system 110 includes a first retainer sheath 120 and a second retainer sheath 122 that are fitted over the different outer ends of the prosthesis 22. In many aspects, the retainer sheaths 120, 122 are similar to the retainer sheath 20 described herein. Each of the retainer sheaths 120, 122 can include a tubular body extending between the outer ends 126, 127 and the inner ends 128, 129, respectively. The splitting members 140,141 are coupled to the respective retainer sheaths 120, 122. The splitting members 140, 141 can have the first outer end portions 142,143 and the second inner end portions 144, 145, respectively, extending from the ends of the respective retainer sheaths. The intermediate portions 146, 147 can be disposed between the respective end portions of the corresponding splitting member.

FIG. 4B illustrates one of the retainer sheaths 122 removed from the corresponding end 34 of the prosthesis 22 for the expansion thereof, and the other retainer sheath 120 in the process of being split. The outer end portion 142 of the splitting member 140 can be retracted toward the middle M of the prosthesis 22 from the outer end 126 longitudinally along the outside of the prosthesis, which may contact the vessel wall when being pulled out. The splitting member may be further retracted outwardly away from the prosthesis in the radial direction of arrow A of the trauma pathway. In other words, the outer end portion 142 can be moved toward the inner end portion 144 in order to split or cut through the wall of the retainer sheath to form a split from the outside-in direction between the ends of the retainer sheath. It is recognized that the inner end portion 144, simultaneously or sequentially, may also be moved toward the outer end portion 142 to split the wall of the retainer sheath from the inside-out direction. When retracted, the splitting member 140 can split or cut the wall of the retainer sheaths 120, 122 to allow for expansion, either simultaneous or sequential expansion, of the outer ends of the prosthesis 22 to the expanded configuration for engagement with a body vessel wall before the expansion of the intermediate segment 36 of the prosthesis 22.

The outer ends 126, 127 of the respective retainer sheaths 120, 122 and the corresponding outer ends the prosthesis 22 can be coextensive or different from each other. For example, FIG. 5 depicts the axial length of the retainer sheaths can be sufficient for the outer end of the retainer sheaths to extend beyond the outer ends of the prosthesis 22 by about the distance X (see FIG. 2). Each of the retainer sheaths 120, 122 can have the first slits 150, 151 and the second slits 152, 153 formed in the wall of the respective retainer sheath.

FIGS. 5-7 depict the system 110 provided with an inner barrier segment 170 disposed within the retainer sheath between the prosthesis and the splitting member. It can be appreciated by those skilled in the art that the delivery system 10 may also include the barrier segment. The barrier segment 170 can prevent the prosthesis 22, such as the graft body or the support structure, from being compromised or otherwise damaged with the movement of the splitting member during the cutting action. The barrier segment 170 includes a body 172 extending axially between a first end 174 and a second end 176. The length of the barrier segment 170 can be sufficient to extend at least partially between the outer and inner ends of each of the retainer sheaths 120, 122 so that each retainer sheath is provided with its own barrier segment. In such arrangement, the first end of the barrier segment can extend to the end of the slit or may extend short of the anchoring member 54 in order to provide clearance for the anchoring member to engage the vessel wall. As shown in FIG. 5, a single barrier segment can extend approximately between the outer ends of the first and second retainer sheaths 120, 122.

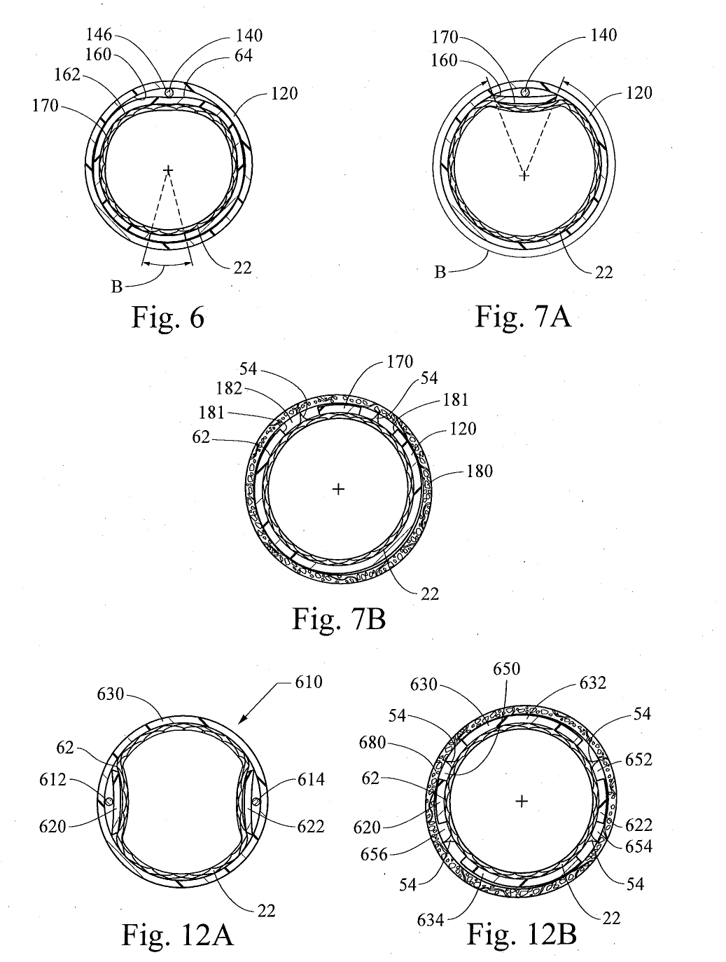

The barrier segment 170 can have many configurations. In one example, the barrier segment can be a tubular sleeve. In another example, the barrier segment can be a pre-split sheath. The barrier segment 170 may have a slit extending completely between its first and second ends 174, 176. The width of the slit can be negligible, i.e., the confronting edges defined by the slit may be in an abutting relationship, so that the barrier segment covers about 360 degrees of the circumference of the prosthesis. The pre-split sheath may be sized such that the confronting edges of the slit overlap one another. In another example, the width of the slit can be larger, shown as a distance B. FIG. 6 illustrates the distance B is about 1 to 3 mm wide so that the barrier segment 170 covers a substantial portion (e.g., about 300 degrees up to 360 degrees) of the circumference of the prosthesis. FIG. 7A illustrates the distance B being relatively larger to form a strip of material having a width sized to cover the splitting member, such as about 1 to 3 mm wide strip, so that the barrier segment 170 covers a relatively smaller portion (e.g., up to about 60 degrees) of the circumference of the compressed prosthesis.

The barrier segment 170 can be configured to permit immediate expansion of the prosthesis once the retainer sheath is removed. The barrier segment 170 may have a configuration with an intermediate portion similar to the cross section in FIG. 6 and strips extending outward from the intermediate portion with a smaller cross-section, such as shown in FIG. 7A, that is relatively smaller than the intermediate portion. This alternative configuration may provide greater clearance for the prosthesis to engage the vessel wall directly for improved initial fixation with the vessel wall.

The relative circumferential position between the splitting member (e.g., the slits of the retainer sheath 120) and the slit of the barrier segment 170, as well as the relative size between the barrier segment and the retainer sheath can be selected to increase the risk of surface area contact between the expanded prosthesis and the vessel wall. FIG. 7B illustrates the prosthesis 22 with the delivery system configuration of FIG. 7A now radially expanded within a vessel portion 180. This is a result of the splitting member 140 splitting through the wall of the retainer sheath 120 such that the formed split edges 181 of the retainer sheath separate from one another, likely from the expansion of the prosthesis, to form a separated region 182. The barrier segment 170 can be sized and positioned to fit within the separated region 182, preferably forming open areas between the barrier segment 170 and the edges 181 of the retainer sheath 120. Such open areas permit portions of the outer surface 62 of the prosthesis 22 to contact the vessel portion 180 directly. For example, when the anchoring member 54 is provided on the prosthesis 22, portions of the anchoring member may extend within the open areas for fixation with the vessel portion 180 to prevent migration or translation of the prosthesis from the vessel portion when the barrier segment and the retainer sheath are removed. Different configurations between the barrier segment and the retainer sheath can provide multiple open areas in order to increase the surface area contact between the prosthesis and the vessel wall.

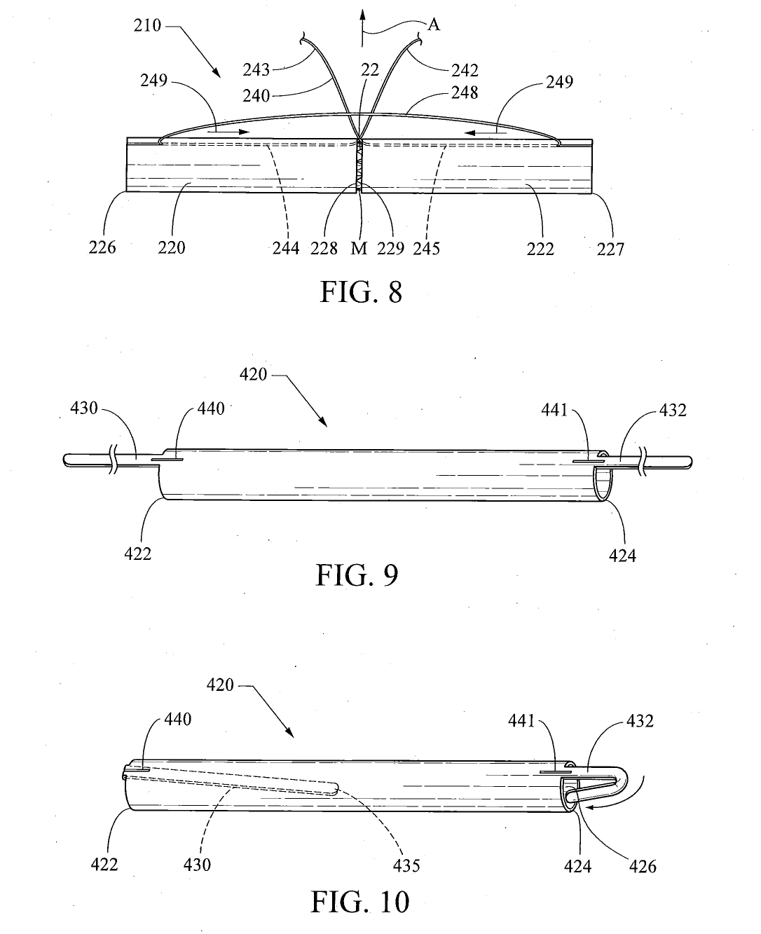

FIG. 8 illustrates another example of a deployment system 210 where the splitting member has an alternative configuration. The deployment system 210 may include a first retainer sheath 220 and a second retainer sheath 222 that are fitted over the different outer ends of the prosthesis 22. In many aspects, the retainer sheaths 220, 222 are similar to the retainer sheaths 20, 120, 122 described herein. For instance, each of the retainer sheaths 220, 222 can include a tubular body extending between the outer ends 226, 227 and the inner ends 228, 229, respectively. The splitting member 240 can have the first and second outer end portions 242, 243 extending from the inner ends 228, 229 of the respective retainer sheaths, i.e., the outer end portions extend generally from the middle M of the prosthesis. First and second inner portions 244, 245 (shown in phantom lines) can extend longitudinally outward from the respective outer end portions 242, 243. A third outer end portion 248 interconnects the first and second inner portions 244, 245 and extends along the outside of the retainer sheaths.

Retraction of the outer end portions 242, 243 can occur in the radial direction A away from the retainer sheaths at the middle M of the prosthesis to split or cut the retainer sheaths from the outside-in direction. For instance, as the outer end portion 242 is retracted, the first inner portion 244 translates along the inside of the retainer sheath to pull a length of the third outer end portion 248 into the retainer sheath. As a result, the pulled length of the third outer end portion 248 of the splitting member performs a splitting action through the wall of the retainer sheath in the direction of the arrow 249. One advantage of the system 210 is that the pulling action of the outer end portion of the splitting member results in movement of the splitting member being contained within the retainer sheath, rather than along the outside the retainer sheath, which may be less invasive to the vessel wall. Moreover, the pulling action can be directly in the radial direction A through the trauma pathway, instead of a combination of directions such as in a longitudinal direction along the outside of the prosthesis and the radial direction A.

It is contemplated that the system 210 may include a single retainer sheath over the entire prosthesis, similar to the sheath 20; however, an opening can be formed in the middle of the retainer sheath to allow the passage of the outer end portions of the splitting member. Here, one outer end portion of the splitting member can be withdrawn relative to the other to release one of the ends of the prosthesis for expansion, and vice versa for the other end of the prosthesis. The outer end portions can be pulled simultaneously and/or sequentially to selectively control the expansion of the prosthesis.

FIGS. 9-10 illustrate an alternative outer retainer sheath embodiment that can be used for any of the systems described herein. The retainer sheath 420 can include one or more tabs extending from one or both outer ends of the retainer sheath. The tab can be configured to function similar to the inner barrier segment 170 of FIGS. 4-7. One advantage of the retainer sheath with the fixed tab is that the steps of loading and positioning a separate inner member, such as the barrier segment 170, within the retainer sheath can be avoided during manufacturing. This would also eliminate the step for removing the inner member during deployment.

FIG. 9 depicts a first tab 430 extending longitudinally outward from the first outer end 422 of the retainer sheath 420, and a second tab 432 extending longitudinally outward from the second outer end 424 of the retainer sheath. The tabs 430, 432 can be a length of material that is attached to the retainer sheath in a separate step, or is an extension of the material that forms the retainer sheath. The tabs can have many configurations. Although the tabs 430, 432 can be a longitudinal strip as shown in FIGS. 9-10, the tabs can be wider to a degree where the tabs have a tubular configuration. The width of the tabs, such as at least about 1 mm to about 3 mm, can be sufficient to shield the prosthesis from the splitting member. The tabs at each end may also have a different configuration from one another if desired.

At least one slit can be formed in the retainer sheath, similar to the slits 50, 52 in FIG. 1, to allow the passage of the splitting member, which resides inside the retainer sheath and is to be extended external the retainer sheath through the slit. Slits 440, 441 can be formed in the wall of the retainer sheath 420 and are preferably located in alignment with the location of the tabs 430, 432. In one example, the slits 440, 441 can extend outwardly beyond the respective outer ends 422, 424 of the retainer sheath 420 and partially within the respective tabs 430, 432. The tab with the slit can inhibit relative rotation between the tab and the retainer sheath during manufacturing to ensure that the tab remains aligned with the slit.

FIG. 10 illustrates that the tabs 430, 432 can be inverted or folded into the lumen 426 of the retainer sheath 420, typically prior to insertion of the splitting member. In this configuration, the tabs are to be disposed between the outer surface of the prosthesis and the internal portion of the splitting member, and the slit may overlap itself in order to permit the passage of the splitting member. The length of the tabs 430, 432 can be sufficient so that the end 435 of the tabs can be located anywhere between the opposite outer end (i.e., extending substantially the entire length of the retainer sheath) and about halfway through the retainer sheath (shown if phantom lines). In one example, the ends of the tabs can be in an abutting relationship so that the tabs in combination form a barrier along the entire inside of the retainer sheath. It is contemplated that the retainer sheath can have a single tab located at one of the outer ends of the retainer sheath, which when folded into the retainer sheath may extend up to the full length of the retainer sheath.

The outer retainer sheaths and/or the barrier segment described herein can be constructed from one or more biocompatible polymeric layers. It is desirable that the sheath and the barrier segment are made from materials that are thin as possible to reduce the overall delivery profile of the system. For example, the sheath and/or segment can be extruded from a biocompatible polymer material. In addition, the sheath and/or segment can be formed of at least one layer such as a polyether block amide, nylon, polyurethane, polytetrafluoroethylene (PTFE), FEP, or any combination thereof. The sheath and/or the barrier segment can be configured to be separated, preferably longitudinally, along a relatively predictable path. The material of the retainer sheath is configured to be split or cut into two or more portions by movement of the splitting member, thereby opening a fissure along the length that permits its removal from around the prosthesis situated therein. A predetermined split line may be formed in the sheath and/or the barrier segment through which the tear or split progresses due to properties of, and/or features incorporated into the material. When present, the predetermined split line can withstand being subjected to a curve to the degree required by the particular application without kinking or premature separation. In one example, the sheath can comprise a splittable polymer such as molecularly oriented, non-isotropic PTFE that is used to make the PEEL-AWAY® Introducer Sheath, which is commercially provided by Cook Medical Inc. (Bloomington, Ind.). Such sheath is described in, e.g., U.S. Pat. No. 4,306,562 to Osborne and U.S. Pat. No. 4,581,025 to Timmermans, each of which is incorporated herein by reference in its entirety. In other examples, the sheath can include one or more preweakened features, such as a score line, perforations, or reduced wall thickness regions, extending longitudinally along the length of the sheath.

The prosthesis 22 can be any type of implant, stent, graft or conduit that is used for medical applications, and an exemplary prosthesis is shown in the figures. The prosthesis can include a generally tubular graft portion and one or more stent structures that are attached to the graft. The prosthesis can be expandable between the radially compressed, delivery configuration that is shown in FIGS. 1 and 4, to the radially expanded, deployed configuration. The stent structure can be attached to an outer surface of the graft so that a lumen of the graft may provide a clear path for fluid flow, and/or attached to the inner surface of the graft. The prosthesis can be sized and shaped for suitable placement within a body vessel, such as an artery or vein, and most particularly, for placement at the site of a vascular trauma such as a transected vessel. The stent structure can be any pattern of stent structures in the art that are designed primarily for vascular applications, and can be self-expanding or balloon expandable. The anchoring member, such as the anchoring member 54, can be disposed along any portion of the prosthesis for securely engaging the vessel wall in order to inhibit migration of the prosthesis after deployment or detachment of the vessel wall from the prosthesis. Preferably, the anchoring member is disposed along the two end portions of the prosthesis. The anchoring member can include barbs or various shaped member structures, including fibers, bristles, or outer protruding and penetrable media. Preferably, the anchoring member provides vessel fixation to the wall tissue, while avoiding adverse conditions associated with disturbing the vasa vasorum and/or pressure induced necrosis of the medium muscular arteries of the type that may result from tying ligatures circumferentially around a connector or a vascular conduit. For example, the anchoring member may be sized and shaped to penetrate the tunica intima, the basement membrane, and partially into the tunica media of a typical body vessel wall, and preferably does not enter the tunica adventitia, and more importantly, do not disturb or otherwise adversely affect the vasa vasorum.

The graft can be a liner that extends at least entirely along the luminal wall of stent structure. The graft can, be made of material to inhibit fluid or blood located within the prosthesis lumen from passing through the graft. In other words, fluid flow is urged by the graft to enter into one end and exit out of the end of the prosthesis. The graft can be formed from conventional materials well known in the medical arts. It is preferred that the graft covering have a porosity for sufficient capillarization and be relatively thin as possible (e.g., about 0.005 inches to about 0.010 inches, and preferably about 0.001 to about 0.0035 inches). Examples of pore density and pore size for the graft covering, as well as other types of materials for a graft covering can be found in U.S. Pat. No. 7,244,444 to Bates, which is incorporated herein by reference in its entirety. A particularly preferred material is expanded polytetrafluoroethylene (ePTFE). Other materials that may be suitable in a particular case include, among others, polytetrafluoroethylene, silicone, polyurethane, polyamide (nylon), as well as other flexible biocompatible materials. Graft covering 15 can also be formed from known fabric graft materials such as woven polyester (e.g. DACRON®), or from a bioremodelable material. One exemplary graft material is THORALON® from Thoratec Corporation (Pleasanton, Calif.), that can prevent leakage of fluid through the pores of the graft. THORALON® is a polyetherurethane urea blended with a siloxane containing surface modifying additive, and has been demonstrated to provide effective sealing of textile grafts. Another example is polyethylene, and in particular, an ultra-high molecular weight polyethylene (UHMwPE), commercially available as DYNEEMA®. The graft may also include a bioremodelable material that can provide an extracellular matrix that permits, and may even promote, cellular invasion and ingrowth into the material upon implantation. Non-limiting examples of suitable bioremodelable materials include reconstituted or naturally-derived collagenous materials. Suitable collagenous materials may include an extracellular matrix material (ECM) that possesses biotropic properties, such as submucosa, renal capsule membrane, dermal collagen, dura mater, pericardium, fascia lata, serosa, peritoneum or basement membrane layers. Suitable submucosa materials may include, for example, intestinal submucosa, including small intestinal submucosa (SIS), stomach submucosa, urinary bladder submucosa, and uterine submucosa. One non-limiting example of a suitable remodelable material is the SURGISIS® BIODESIGN™, which is commercially available from Cook Medical Inc. (Bloomington, Ind.). Another suitable remodelable material is the graft prosthesis material described in U.S. Pat. No. 6,206,931 to Cook et al., incorporated herein by reference. The remodelable material can be ECM, SIS, remodelable or collagenous foam, foamed ECM, lyophilized SIS, vacuum pressed SIS, or the like.

The prosthesis can also include a coating of one or more therapeutic agents along a portion of the conduit body and/or the graft coverings. Therapeutic agents for use as biocompatible coatings are well known in the art. Non-limiting examples of suitable bio-active agents that may be applied to the vascular conduit include thrombo-resistant agents, antibiotic agents, anti-tumor agents, antiviral agents, anti-angiogenic agents, angiogenic agents, anti-mitotic agents, anti-inflammatory agents, angiostatin agents, endostatin agents, cell cycle regulating agents, genetic agents, including hormones such as estrogen, their homologs, derivatives, fragments, pharmaceutical salts and combinations thereof. Those skilled in the art will appreciate that other bioactive agents may be applied for a particular use. The bioactive agent can be incorporated into, or otherwise applied to, portions of the vascular conduit by any suitable method that permits adequate retention of the agent material and the effectiveness thereof for its intended purpose.

In one example of making the delivery system, the retainer sheath is formed from PTFE tubing having an outer diameter of about 0.134 inches and an inner diameter of about 0.124 inches is cut to about 2 cm in length greater than the length of the prosthesis, or 7 cm for a 5-cm prosthesis. Slits are formed in the tubing about 1 cm from the ends. The barrier is formed from PTFE tubing having an outer diameter of about 0.134 inches and an inner diameter of about 0.124 inches is cut to about the same length of the prosthesis, or 5 cm. About a 3-mm longitudinal split is formed along the length of the tubing. The barrier segment is inserted within the retainer sheath to form a subassembly. The split in the barrier segment and the slits in the retainer sheath can be aligned in a manner to permit the most contact area between the prosthesis and the vessel wall when expanded. The splitting member formed from about a 0.010-inch diameter copper wire can be inserted between the barrier segment and the retainer sheath so that ends of the wire extend outwardly from the slits formed in the retainer sheath. A prosthesis is compressed and inserted into the opening of the barrier segment of the subassembly to form the delivery system. When more than one retainer sheath and/or barrier segment subassemblies are present, the different portions of the compressed prosthesis are inserted within the corresponding subassemblies.

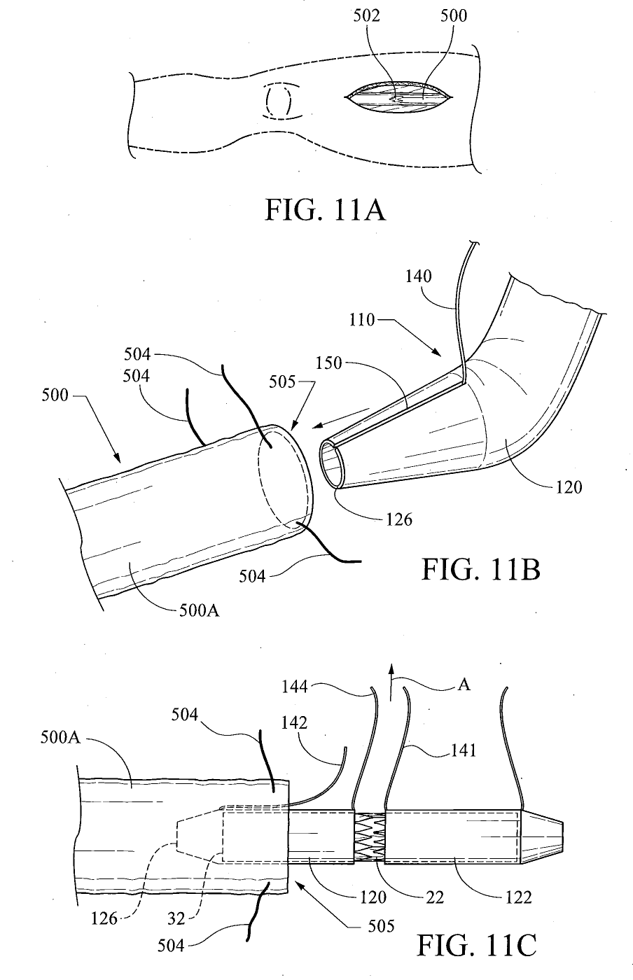

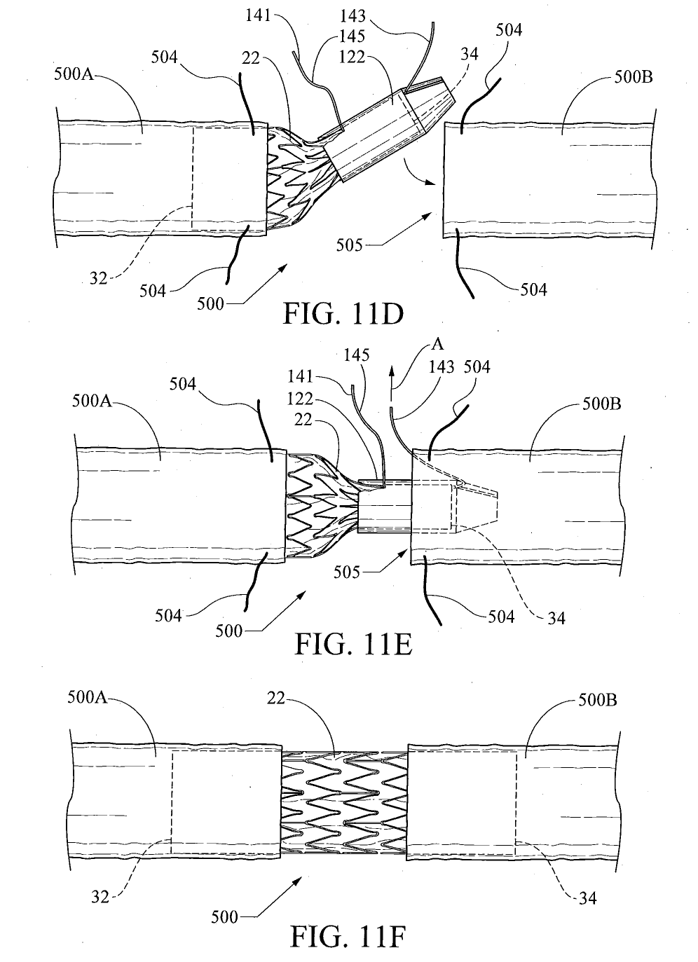

FIGS. 11A-11F illustrate a method of delivering a prosthesis to a transected body vessel to interconnect the two vessel portions during open surgery. In FIG. 11A, a body vessel 500, for example in the leg of a patient, has previously been subjected to a traumatic episode, which results in a portion 502 of body vessel 500 being torn away or otherwise severely damaged. Pre-surgery preparation has been applied to the leg and a trauma pathway may be formed therein in order to gain access to the body vessel and the damaged portion thereof. After clamping the body vessel 500 on both ends of the portion 502 to restrict blood flow temporarily, the blood vessel 500 can be cut or transected into two portions 500A, 500B by the clinician. The transection may be at the damaged portion 502 of the vessel 500 or as far away as necessary from the damaged portion to remove unhealthy portions of the body vessel or unrepairable portions of the body vessel. Sutures 504 can be attached to the end openings 505 of body vessel portions 500A, 500B to keep them fixed in place and opened to facilitate insertion of the prosthesis. Forceps may also be used in a similar manner. Any number of sutures can be used to retain the end openings 505 in the open position, although triangulation sutures can be sufficient, with each suture being about 120 degrees apart from the adjacent suture. A prosthesis is selected to have a radial expanded cross-section and a longitudinal length sufficient to bridge the body vessel portions 500A, 500B and radially fit within the body vessel portions.

In FIGS. 11B-11C, the prosthesis 22, which is preloaded within the deployment device 110, is shown being situated and oriented adjacent the body vessel portion 500A through the trauma pathway. The prosthesis is retained in the delivery, radially compressed configuration by the outer retainer sheath 120.

In FIG. 11B, the outer end 126 of deployment system 110 can be introduced into the end opening 505 of the first portion 500A of transected body vessel 500. The deployment system 110 preferably has the tapered outer end, and can be positioned within the first portion 500A of transected body vessel 500 so that at least a portion of the prosthesis 22 is within the first portion 500A of the body vessel. It is preferred that the vessel portion initially selected be the non-blood supplying vessel end. The vessel portion 500A may be manually pulled over the outer end 126 of the outer retainer sheath 120.

In FIG. 11C, the first outer end 32 of the prosthesis can be inserted into vessel portion 320A by a sufficient distance for the purposes of anchoring. After insertion, the outer portion 142 of the splitting member 140 is positioned to exit out of the end opening 505 of the body vessel portion 500A such that the outer portion 142 is accessible by the clinician through the trauma pathway. The clinician can retract longitudinally the outer portion 142 of the splitting member 140 against the outer surface toward the middle of the prosthesis from the outer end 126 in a manner to avoid excessive contact between the vessel wall and the splitting member. The splitting member may be further retracted outwardly away from the prosthesis in the radial direction A. When retracted, the splitting member 140 can split or cut through the wall of the retainer sheath 120 to allow for expansion of the outer end 32 of the prosthesis 22 to the expanded configuration for engagement with the vessel wall prior to the expansion of the intermediate portion of the prosthesis 22. The splitting member 140 can be removed from the body. After expansion of the prosthesis outer end, the retainer sheath 120 in a split configuration, and the barrier segment (when present), can be removed from the body vessel portion 500A by the clinician by hand or with a hemostat or tweezers. When present, the barrier segment may be initially withdrawn from the vessel portion prior to the retainer sheath in the split configuration since the barrier segment is typically smaller than the retainer sheath and can allow for additional surface area contact between the prosthesis and the vessel wall before withdrawing the larger retainer sheath.

FIGS. 11D-11E illustrate a first portion of the prosthesis in the expanded configuration and implanted within the first body vessel portion 500A after removal of the components of the deployment system 110. The other portion of the prosthesis 22 is depicted in the compressed configuration within the second outer retainer sheath 122, which can remain outside of both of the vessel portions. The deployment device 110 and the prosthesis 22 can then be manipulated by the clinician in order to introduce the second outer end 34 of the prosthesis 22 into vessel portion 500B by a sufficient distance for the purposes of anchoring. The second outer retainer sheath 122 can be cut with retraction of the splitting member 140 such as described above with reference to FIG. 11C. Once a split is formed into the retainer sheath 122, the second end 34 can be expanded to the expanded configuration to engage along the wall tissue of the second portion 500B.

FIG. 11F illustrates the remaining portion of the prosthesis in the expanded configuration and implanted within the second body vessel portion 500B after removal of the components of the deployment system 110. The prosthesis 22 is now fully deployed to interconnect the first and second portions 500A, 500B of the transected body vessel to form a passageway for blood flow. The sutures 504 can then be removed. Preferably, portions of the exterior surfaces of the prosthesis sealably engages with the luminal walls of the body vessel to inhibit leakage of blood and to force blood to flow throughout the body vessel during emergency surgery, and particularly to obtain hemostasis while maintaining blood perfusion. The prosthesis can be permanently placed within the patient, thereby obviating a need for subsequent surgical intervention.

When a single retainer sheath retains the prosthesis in the compressed configuration, such as the delivery system 10 shown in FIG. 1, one or both ends 32, 34 of the prosthesis 22 can be introduced to the vessel portions 500A, 500B prior to full expansion. The ends 42, 44 of the splitting member 40 can be retracted similar to description of FIG. 11C in order to allow for expansion of the ends of the prosthesis within the vessel portions.

It is contemplated that any one of the systems 10, 110, 210 can include a plurality of splitting members, as well as may include additional slits, that are arranged circumferentially offset from one another, such as, e.g., about 30 degrees to about 180 degrees apart. Additional splitting members may permit quicker removal of the retainer sheath and thus faster engagement between the prosthesis and the vessel wall. FIGS. 12A-12B illustrate one example of an alternative deployment system 610 with two splitting members 612, 614. In FIG. 12A, the two splitting members 612, 614 are positioned on opposite sides of the prosthesis 22 in the compressed configuration. The barrier segments 620, 622 are disposed between different portions of the prosthesis 22 and the splitting members 612, 614, respectively. The retainer sheath 630 can maintain the relative positions of the prosthesis in the compressed configuration, the barrier segments, and the splitting members. The two splitting members can cut through the retainer sheath 630 along two split lines so that the retainer sheath 630 is separated into two portions 632, 634, as shown in FIG. 12B. Depending on the relative location of the splitting members, the two portions can be substantially equal or can be sized to be different.

FIG. 12B illustrates the prosthesis 22 of FIG. 12A now expanded within a vessel portion 680. When expanded, open areas, such as, e.g., four open areas 650, 652, 654, 656 between the barrier segments 620, 622 and the separated edges of the retainer sheath portions 632, 634 permit portions of the outer surface 62 of the prosthesis 22 to contact the vessel portion 680. When the anchoring member 54 is provided on the prosthesis 22, portions of the anchoring member may extend within the open areas 650, 652, 654, 656 for fixation with the vessel portion 680 to prevent migration or translation of the prosthesis from the vessel portion when the barrier segment and the retainer sheath are removed. It is desirable that the four areas be located at different quadrants so that the surface area contact between the expanded prosthesis and the vessel wall is distributed along those areas.

Although the prosthesis and the deployment system has been described in connection with its primary intended use for repair of vascular trauma, those skilled in the art will appreciate that the device may also be used to repair other traumatic conditions. Non-limiting examples of such conditions include aneurysms, such as abdominal aorta aneurysms, and surgery for tumor removal. In another matter of terminology there are many types of body canals, blood vessels, ducts, tubes, and other body passages, and the term “body vessel” is meant to include all such passages. Other vascular applications include coronary arteries, carotid arteries, vascular aneurysms, and peripheral arteries and veins (e.g., renal, iliac, femoral, popliteal, subclavian, aorta, intracranial, etc.). Other nonvascular applications include gastrointestinal, duodenum, biliary ducts, esophagus, urethra, reproductive tracts, trachea, and respiratory (e.g., bronchial) ducts. To this end, the deployment systems and methods described herein can be used to deliver a prosthesis to any of these vessels, ducts, canals, tubes or body passageways.

Drawings in the figures illustrating various embodiments are not necessarily to scale. Some drawings may have certain details magnified for emphasis, and any different numbers or proportions of parts should not be read as limiting, unless so-designated in the present disclosure. Those of skill in the art will appreciate that embodiments not expressly illustrated herein may be practiced within the scope of the present invention, including those features described herein for different embodiments may be combined with each other and/or with currently-known or future-developed technologies while remaining within the scope of the claims presented here. It is therefore intended that the foregoing detailed description be regarded as illustrative rather than limiting. And, it should be understood that the following claims, including all equivalents, are intended to define the spirit and scope of this invention.

We claim:

1. A deployment system for repair of a body vessel comprising:
a prosthesis having a first prosthesis end and a second prosthesis end, and being radially movable between a compressed configuration and an expanded configuration;
a retainer sheath fitted at least partially over a segment of the prosthesis to retain said segment in the compressed configuration, the retainer sheath comprising a tubular body, a first sheath end, a second sheath end, and a lumen extending therethrough to receive the prosthesis;
a splitting member having an internal portion disposed between a luminal wall of the retainer sheath and the prosthesis, and an external portion disposed external to the retainer sheath, wherein the splitting member is operable to split a wall of the retainer sheath along a direction toward a middle of the prosthesis away from at least one of the first and second sheath ends upon retraction of the external portion of the splitting member, wherein in response to being split by the splitting member, the retainer sheath has a split configuration and the corresponding prosthesis end is allowed to move to the expanded configuration for engagement with a body vessel wall.

2. The system of claim 1, wherein the external portion of the splitting member comprises a first end portion and a second end portion, and the internal portion comprises an intermediate portion connected between the first and second end portions, wherein:
in response to retraction of the first end portion, the wall of the retainer sheath is split in a first direction toward the middle of the prosthesis away from the first sheath end to permit expansion of the first prosthesis end; and
in response to retraction of the second end portion, the wall of the retainer sheath is split in a second direction, opposite the first, toward the middle of the prosthesis away from the second sheath, end to permit expansion of the second prosthesis end.

3. The system of claim 2, wherein the retainer sheath has a slit extending from at least one of the first and second ends toward the middle of the retainer sheath, the slit configured to permit passage of the corresponding end portion of the splitting member therethrough.

4. The system of claim 1, further comprising an inner barrier segment disposed between the internal portion of the splitting member and an outside wall of the prosthesis, extending axially at least partially between the first and second sheath ends.

5. The system of claim 4, wherein the barrier segment comprises a segment width along the circumference of the prosthesis in the expanded configuration, and the retainer sheath in the split configuration comprises a sheath width along the circumference of the prosthesis in the expanded configuration, the segment width and the sheath width dimensioned and arranged to permit an open circumferential area between the barrier segment and the retainer sheath and allow direct contact between the prosthesis in the expanded configuration and the body vessel wall.

6. The system of claim 4, wherein at least one of the first and second sheath ends comprises a tab member that is folded and disposed within the lumen of the retainer sheath to form the inner barrier segment.

7. The system of claim 4, wherein the retainer sheath extends at least partially beyond each of the first and second prosthesis ends and ends of the barrier segment.

8. The system of claim 1, wherein the splitting member is a first splitting member operable to split a first portion of the retainer sheath proximate the first sheath end, and the system further comprises:
a second splitting member having an internal portion disposed between the luminal wall of the retainer sheath and the prosthesis, and an external portion disposed external to the retainer sheath, wherein the second splitting member is operable to split a wall of a second portion of the retainer sheath in a direction toward a middle of the prosthesis away from the second sheath end upon retraction of the external portion of the second splitting member.

9. The system of claim 1, wherein the retainer sheath further comprises a first retainer sheath and a second retainer sheath, the first retainer sheath fitted over a first portion of the prosthesis proximate the first prosthesis end, the second retainer sheath fitted over a second portion of the prosthesis proximate the second prosthesis end.

10. The system of claim 9, wherein the splitting member comprises a first splitting member and a second splitting member, the first splitting member associated with the first retainer sheath, and the second splitting member associated with the second retainer sheath.

11. The system of claim 1, wherein the external portion of the splitting member is retractable along an outer surface of the retainer sheath relative to the internal portion of the splitting member that remains in a fixed position.

12. The system of claim 1, wherein the internal portion of the splitting member is retractable along an inner luminal surface of the retainer sheath relative to the external portion of the splitting member that remains in a fixed position.

13. A deployment system for repair of a body vessel comprising:
a prosthesis having a first prosthesis end and a second prosthesis end, and being radially movable between a compressed configuration and an expanded configuration;
a retainer sheath fitted at least partially over a segment of the prosthesis to retain said segment in the compressed configuration, the retainer sheath comprising a tubular body, a first sheath end, a second sheath end, and a lumen extending therethrough to receive the prosthesis;
a splitting member having an internal portion disposed within the retainer sheath and an external portion disposed external to the retainer sheath; and
an inner barrier segment disposed between the internal portion of the splitting member and an outside wall of the prosthesis, extending axially at least partially between the first and second sheath ends, wherein the splitting member operable to split a wall of the retainer sheath along a direction toward a middle of the prosthesis away from at least one of the first and second sheath ends upon retraction of the external portion of the splitting member to allow portions of the prosthesis to move to the expanded configuration.

14. The system of claim 13, wherein the retainer sheath is a first retainer sheath fitted over the first prosthesis end, and the system further comprises a second retainer sheath fitted over the second prosthesis end, each of the first and second retainer sheaths having an outer sheath end and an inner sheath end.

15. The system of claim 14, wherein the splitting member is a first splitting member associated with the first retainer sheath, and the system further comprises a second splitting member associated with the second retainer sheath, wherein the first and second splitting members are operable to split the wall of the retainer sheath along the direction toward the middle of the prosthesis away from the respective outer sheath ends upon retraction of the external portion of the corresponding splitting member.

16. The system of claim 14, wherein the inner barrier segment is a first inner barrier segment associated with the first retainer sheath, and the system further comprises a second inner barrier segment associated with the second retainer sheath,
wherein each of the first and second inner barrier segments comprises a segment width along the circumference of the prosthesis in the expanded configuration, and the retainer sheath in the split configuration comprises a sheath width along the circumference of the prosthesis in the expanded configuration, the segment width and the sheath width dimensioned and arranged to permit an open circumferential area between the barrier segment and the retainer sheath and allow direct contact between the prosthesis in the expanded configuration and the body vessel wall.

17. A method of interconnecting a first vessel portion and a second vessel portion of a transected body vessel, comprising:
introducing a first end of a prosthesis retained in a compressed configuration by a retainer sheath in a first vessel portion, wherein a splitting member is associated with the retainer sheath and is operable to split a wall of the retainer sheath; and
retracting a portion of the splitting member in a direction away from a middle of the prosthesis to split the retainer sheath from an outer end thereof and toward the middle such that the first end of the prosthesis is permitted to expand to an expanded configuration for engagement with a vessel wall of the first vessel portion.

18. The method of claim 17, wherein an inner barrier segment is disposed between an internal portion of the splitting member and an outside wall of the prosthesis, extending axially at least partially between outer ends of the retainer sheath.

19. The method of claim 17, further comprising:
introducing a second end of the prosthesis retained in a compressed configuration by a second retainer sheath in a second vessel portion, wherein a second splitting member is associated with the second retainer sheath and is operable to split a wall of the second retainer sheath; and
retracting a portion of the second splitting member in a direction away from a middle of the prosthesis to split the second retainer sheath from an outer end thereof and toward the middle such that the second end of the prosthesis is permitted to expand to an expanded configuration for engagement with a vessel wall of the second vessel portion.

20. The method of claim 17, wherein the first end of the prosthesis is retained in the compressed configuration by a first retainer sheath, and the second end of the prosthesis is retained in the compressed configuration by a second retainer sheath.
